(19) United States
(12) Patent Application Publication
(10) Pub. No.: US 2026/0250240 A1
(43) Pub. Date: Aug. 27, 2026

(54) PHOTOCHEMICAL SYSTEMS AND METHODS FOR PREPARING CYANOBENZAMIDES

(71) Applicants: FMC CORPORATION, Philadelphia, PA (US); FMC IP TECHNOLOGY GMBH, Neuhausen (CH)

(72) Inventors: Jianhua MAO, Columbus, OH (US); Hao WANG, Shanghai (CN); Yihui XU, Shanghai (CN)

(21) Appl. No.: 19/161,439

(22) PCT Filed: Feb. 27, 2024
(86) PCT No.: PCT/US2024/017382

§ 371 Date: Sep. 2, 2025

Related U.S. Application Data
(60) Provisional application No. 63/449,694, filed on Mar. 3, 2023.

(51) Int. Cl. C07C 253/04

(52) U.S. Cl. CPC C07C 253/04

(57) ABSTRACT

Described herein is a loop reactor system for performing photochemical reactions. The loop reactor system is particularly advantageous for preparing cyanobenzamides.

Also disclosed are photochemical methods for preparing cyanobenzamides and compositions containing cyanobenzamides using.

### CROSS-REFERENCE TO RELATED APPLICATIONS

This application claims the benefit of U.S. Provisional Application No. 63/449,694, filed Mar. 3, 2023, all of which is incorporated by reference herein in its entirety.

### FIELD

This disclosure is directed to methods and an apparatus to prepare cyanobenzamides. Compounds prepared by the methods disclosed herein are useful for preparation of certain anthranilamide compounds that are of interest as insecticides, such as, for example, the insecticides chlorantraniliprole and cyantraniliprole.

### BACKGROUND

Photochemical reactions are known to be useful as an alternative way to conduct certain chemical reactions. For example, synthesis of electron-donating substituted benzonitriles by photolysis of phenyl halides and esters was described by Dichiarante et al. (Chem. Commun., 2006, 3001-3003). However, these reactions are often limited by the photochemical reactor systems used to conduct them. As a result, these known photochemical reactions often have unoptimized yields and incomplete reactions.

The present disclosure provides a loop reactor system for performing photochemical reactions. The loop reactor system is particularly advantageous for preparing cyanobenzamides. The loop reactor system provides high yields and optimized reactions by continuously cycling a reaction mixture until a satisfactory conversion is achieved.

### BRIEF DESCRIPTION

In one aspect, provided herein is a photochemical reaction system comprising:
a loop reactor system comprising:
   a receiving vessel configured to receive a mixture;
   a photochemical reaction vessel configured to subject the mixture to irradiation from at least one light source; and
   a precipitation vessel configured to receive the irradiated mixture; and
optionally a further reaction system component selected from the group of fluid transfer systems, stripping systems, filtration systems, drying systems, stirring systems, temperature control systems, and combinations thereof.

In another aspect, provided herein is a method of preparing a compound of Formula II

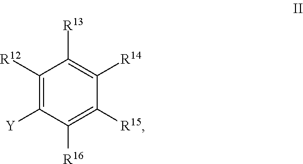

wherein:
R¹² and R¹³ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof;
R¹⁴ is selected from the group of amines, amides, carbamides, NH₂, NR¹⁷R¹⁸, and NR²¹COR²²;
R¹⁵ is selected from the group of amides, C(O)NR¹⁹R²⁰, and C(O)NHCH₃;
R¹⁶, R¹⁷, R¹⁸, R¹⁹, R²⁰, R²¹, and R²² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
Y is selected from the group of cyano-containing substituents and CN,
   the method comprising:
I) forming a mixture comprising:
   a) a compound of Formula I

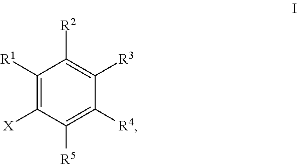

wherein:
R¹ and R² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
R³ is selected from the group of amines, amides, carbamides, NH₂, NR⁶R⁷, and NR⁸COR⁹;
R⁴ is selected from the group of amides, C(O)NR¹⁰R¹¹, and C(O)NHCH₃;
R⁵, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide;
R²³ and R²⁴ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, C₁-C₆ haloalkyl, and C₁-C₆ fluoroalkyl;
   b) a cyano-containing compound; and
   c) optionally a solvent; and
II) subjecting the mixture to irradiation from at least one light source.

In another aspect, provided herein is a composition comprising: (a) a compound of Formula II, and (b) a compound of Formula I.

### BRIEF DESCRIPTION OF THE DRAWINGS

FIG. 1 is an exemplary photochemical reaction system in accordance with the present disclosure.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION OF THE DISCLOSURE

The photochemical reaction system of the present disclosure comprises a loop reactor system comprising: a receiving vessel configured to receive a mixture; a photochemical reaction vessel configured to subject the mixture to irradiation from at least one light source; and a precipitation vessel configured to receive the irradiated mixture. The photochemical reaction system optionally further comprises a further reaction system component selected from the group of fluid transfer systems, stripping systems, filtration systems, drying systems, stirring systems, temperature control systems, and combinations thereof.

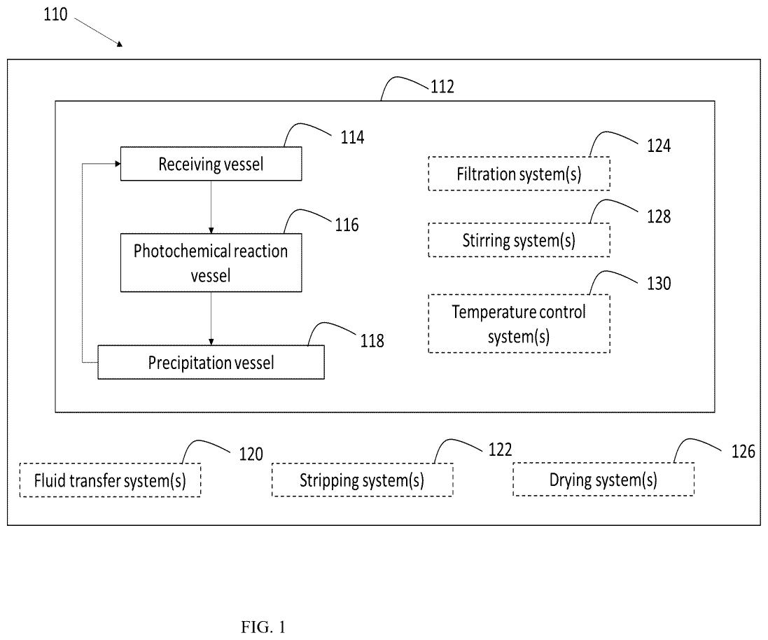

FIG. 1 is an exemplary photochemical reaction system 110. In this exemplary embodiment, photochemical reaction system 110 includes a loop reactor system 112 that includes a receiving vessel 114 configured to receive a mixture, a photochemical reaction vessel 116 configured to receive the mixture from the receiving vessel 114 and then subject the mixture to irradiation from at least one light source, and a precipitation vessel 118 configured to receive the irradiated mixture from the photochemical reaction vessel 116. The arrows indicate the flow of the mixture. As can be seen, the mixture can flow from the precipitation vessel 118 to the receiving vessel 114. Reaction cycling may be achieved with this embodiment. The photochemical reaction system 110 optionally further comprises one or more further reaction system components shown in boxes outlined with dashed lines. These components include fluid transfer system(s) 120, stripping system(s) 122, filtration system(s) 124, drying system(s) 126, stirring system(s) 128, and temperature control system(s) 130. Among these optional components, the stirring system(s) 128, filtration system(s) 124, and temperature control system(s) 130 are included in the loop reactor system 112, while the remainder of these components are separate from the loop reactor system 112 and are used for post-reaction workup.

In some embodiments, the loop reactor system 112 is configured to cycle the mixture. In some embodiments, the loop reactor system 112 is configured to cycle the mixture at least once through the receiving vessel 114, the photochemical reaction vessel 116, and the precipitation vessel 118.

In some embodiments, the photochemical reaction system comprises at least one fluid transfer system. In some embodiments, the photochemical reaction system comprises at least one fluid transfer system configured to: transfer the mixture from the receiving vessel 114 to the photochemical reaction vessel 116; transfer the mixture from the photochemical reaction vessel 116 to the precipitation vessel 118; and/or transfer the mixture from the precipitation vessel 118 to the receiving vessel 114. In some embodiments, the at least one fluid transfer system comprises at least one pump.

In some embodiments, the photochemical reaction system comprises a stripping system 122, a stirring system 128, a filtration system 124, a drying system 126, and/or a temperature control system 130.

In many embodiments, the photochemical reaction vessel is configured to subject the mixture to irradiation from at least one light source. In some embodiments, the photochemical reaction vessel is configured to subject the mixture to irradiation from a plurality of light sources. In some embodiments, the photochemical reaction vessel is configured to subject the mixture to irradiation from two light sources. In some embodiments, the photochemical reaction vessel is configured to subject the mixture to irradiation from three light sources. In some embodiments, the photochemical reaction vessel is configured to subject the mixture to irradiation from four light sources. In some embodiments, the photochemical reaction vessel is configured to subject the mixture to irradiation from more than four light sources.

In many embodiments, the photochemical reaction vessel is configured to subject the mixture to irradiation. Generally, the radiation may be of any suitable wavelength known in the art. In some embodiments, the radiation comprises ultraviolet radiation, visible radiation, or a combination thereof.

In some embodiments, the radiation has a minimum wavelength selected from 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, and 800 nm.

In some embodiments, the radiation has a maximum wavelength selected from 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, and 800 nm.

In some embodiments, the photochemical reaction vessel is configured to subject the mixture to a light radiation having a wavelength in a range of from about 180 nm to about 450 nm, preferably from about 180 nm to about 420 nm, more preferably from about 360 nm to about 420 nm, even more preferably from about 360 nm to about 400 nm.

In some embodiments, at least one of the receiving vessel 114 and the precipitation vessel 118 comprises a filter.

The embodiments of this disclosure include:

Embodiment 1. A photochemical reaction system comprising:
a loop reactor system comprising:
   a receiving vessel configured to receive a mixture;
   a photochemical reaction vessel configured to subject the mixture to irradiation from at least one light source; and
   a precipitation vessel configured to receive the irradiated mixture; and
optionally a further reaction system component selected from the group of fluid transfer systems, stripping systems, filtration systems, drying systems, stirring systems, temperature control systems, and combinations thereof.

Embodiment 2. The photochemical reaction system of Embodiment 1, wherein the loop reactor system is configured to cycle the mixture at least once through the receiving vessel, the photochemical reaction vessel, and the precipitation vessel.

Embodiment 3. The photochemical reaction system of Embodiment 1, wherein the photochemical reaction system comprises at least one fluid transfer system configured to:
transfer the mixture from the receiving vessel to the photochemical reaction vessel;
transfer the mixture from the photochemical reaction vessel to the precipitation vessel; and/or
transfer the mixture from the precipitation vessel to the receiving vessel.

Embodiment 4. The photochemical reaction system of Embodiment 1, wherein the photochemical reaction system comprises a stripping system, a stirring system, a filtration system, a drying system, a temperature control system, or a combination thereof.

Embodiment 5. The photochemical reaction system of Embodiment 1, wherein the photochemical reaction vessel is configured to subject the mixture to irradiation from a plurality of light sources.

Embodiment 6. The photochemical reaction system of Embodiment 1, wherein the photochemical reaction vessel is configured to subject the mixture to a light radiation having a wavelength in a range of from about 180 nm to about 450 nm, preferably from about 180 nm to about 420 nm, more preferably from about 360 nm to about 420 nm, even more preferably from about 360 nm to about 400 nm.

Embodiment 7. The photochemical reaction system of Embodiment 1, wherein at least one of the receiving vessel and the precipitation vessel comprises a filter.

Embodiment 8. A method of using the photochemical reaction system of Embodiment 1 to prepare a compound of Formula II

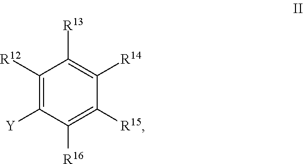

wherein:
R¹² and R¹³ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof;
R¹⁴ is selected from the group of amines, amides, carbamides, NH₂, NR¹⁷R¹⁸, and NR²¹COR²²;
R¹⁵ is selected from the group of amides, C(O)NR¹⁹R²⁰, and C(O)NHCH₃;
R¹⁶, R¹⁷, R¹⁸, R¹⁹, R²⁰, R²¹, and R²² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
Y is selected from the group of cyano-containing substituents and CN,
   the method comprising:
I) providing to the receiving vessel of the photochemical reaction system a mixture comprising:
   a) a compound of Formula I

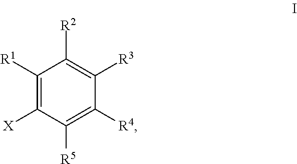

wherein:
R¹ and R² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
R³ is selected from the group of amines, amides, carbamides, NH₂, NR⁶R⁷, and NR⁸COR⁹;
R⁴ is selected from the group of amides, C(O)NR¹⁰R¹¹, and C(O)NHCH₃;
R⁵, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide;
R²³ and R²⁴ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, C₁-C₆ haloalkyl, and C₁-C₆ fluoroalkyl;
   b) a cyano-containing compound; and
   c) optionally a solvent; and
II) reacting the mixture in the photochemical reaction system to produce the compound of Formula II.

Embodiment 8A. The method of Embodiment 8, wherein
R¹² and R¹³ are each individually selected from the group of hydrogen, methyl, ethyl, n-propyl, iso-propyl, n-butyl, iso-butyl, sec-butyl, tert-butyl and n-pentyl;
R¹⁴ is NH₂;
R¹⁵ is selected from the group of C(O)NR¹⁹R²⁰ and C(O)NHCH₃;
R¹⁶, R¹⁹, and R²⁰ are each individually selected from the group of hydrogen, methyl, ethyl, n-propyl, iso-propyl, n-butyl, iso-butyl, sec-butyl, tert-butyl, and n-pentyl;
Y is CN;
R¹ and R² are each individually selected from the group of hydrogen, methyl, ethyl, n-propyl, iso-propyl, n-butyl, iso-butyl, sec-butyl, tert-butyl and n-pentyl;
R³ is NH₂;
R⁴ is selected from the group of C(O)NR¹⁰R¹¹ and C(O)NHCH₃;
R⁵, R¹⁰, and R¹¹ are each individually selected from the group of hydrogen, methyl, ethyl, n-propyl, iso-propyl, n-butyl, iso-butyl, sec-butyl, tert-butyl, and n-pentyl;
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
R²³ and R²⁴ are each individually selected from the group of methyl and ethyl.

Embodiment 8B. The method of Embodiment 8, wherein
R¹² and R¹³ are each individually selected from the group of hydrogen and methyl;
R¹⁴ is NH₂;
R¹⁵ is selected from the group of C(O)NR¹⁹R²⁰ and C(O)NHCH₃;
R¹⁶ is selected from the group of hydrogen and methyl;
R¹⁹, and R²⁰ are each individually selected from the group of hydrogen, methyl, ethyl, n-propyl, iso-propyl, n-butyl, iso-butyl, sec-butyl, tert-butyl, and n-pentyl;
Y is CN;
R¹ and R² are each individually selected from the group of hydrogen and methyl;
R³ is NH₂;
R⁴ is selected from the group of C(O)NR¹⁰R¹¹ and C(O)NHCH₃;
R⁵ is selected from the group of hydrogen and methyl;
R¹⁰, and R¹¹ are each individually selected from the group of hydrogen, methyl, ethyl, n-propyl, iso-propyl, n-butyl, iso-butyl, sec-butyl, tert-butyl, and n-pentyl;
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
R²³ and R²⁴ are each individually selected from the group of methyl and ethyl.

Embodiment 8C. The method of Embodiment 8, wherein
R¹² is hydrogen;
R¹³ is methyl;
R¹⁴ is NH₂;
R¹⁵ is selected from the group of C(O)NR¹⁹R²⁰ and C(O)NHCH₃;
R¹⁶ is hydrogen;
R¹⁹, and R²⁰ are each individually selected from the group of hydrogen, methyl, ethyl, n-propyl, and iso-propyl;
Y is CN;
R¹ is hydrogen;
R² is methyl;
R³ is NH₂;
R⁴ is selected from the group of C(O)NR¹⁰R¹¹ and C(O)NHCH₃;
R⁵ is hydrogen;
R¹⁰, and R¹¹ are each individually selected from the group of hydrogen, methyl, ethyl, n-propyl, and iso-propyl;
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
R²³ and R²⁴ are each individually methyl.

Embodiment 9. The method of Embodiment 8, wherein the compound of Formula II is

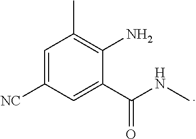

Embodiment 10. The method of Embodiment 8, wherein the compound of Formula I is

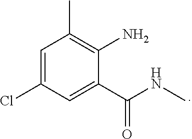

Embodiment 11. A method of preparing a compound of Formula II

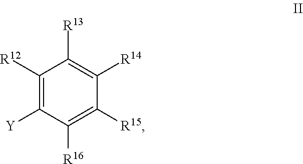

wherein:
R¹² and R¹³ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof;
R¹⁴ is selected from the group of amines, amides, carbamides, NH₂, NR¹⁷R¹⁸, and NR²¹COR²²;
R¹⁵ is selected from the group of amides, C(O)NR¹⁹R²⁰, and C(O)NHCH₃;
R¹⁶, R¹⁷, R¹⁸, R¹⁹, R²⁰, R²¹, and R²² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
Y is selected from the group of cyano-containing substituents and CN,
   the method comprising:
I) forming a mixture comprising:
   a) a compound of Formula I

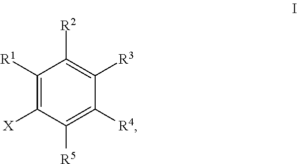

wherein:
   R¹ and R² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
   R³ is selected from the group of amines, amides, carbamides, NH₂, NR⁶R⁷, and NR⁸COR⁹;
   R⁴ is selected from the group of amides, C(O)NR¹⁰R¹¹, and C(O)NHCH₃;
   R⁵, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
   X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide;
   R²³ and R²⁴ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, C₁-C₆ haloalkyl, and C₁-C₆ fluoroalkyl;
   b) a cyano-containing compound; and
   c) optionally a solvent; and
II) subjecting the mixture to irradiation from at least one light source.

Embodiment 11A. The method of Embodiment 11, wherein
R¹² and R¹³ are each individually selected from the group of hydrogen, methyl, ethyl, n-propyl, iso-propyl, n-butyl, iso-butyl, sec-butyl, tert-butyl and n-pentyl;
R¹⁴ is NH₂;
R¹⁵ is selected from the group of C(O)NR¹⁹R²⁰ and C(O)NHCH₃;
R¹⁶, R¹⁹, and R²⁰ are each individually selected from the group of hydrogen, methyl, ethyl, n-propyl, iso-propyl, n-butyl, iso-butyl, sec-butyl, tert-butyl, and n-pentyl;
Y is CN;
R¹ and R² are each individually selected from the group of hydrogen, methyl, ethyl, n-propyl, iso-propyl, n-butyl, iso-butyl, sec-butyl, tert-butyl and n-pentyl;
R³ is NH₂;
R⁴ is selected from the group of C(O)NR¹⁰R¹¹ and C(O)NHCH₃;
R⁵, R¹⁰, and R¹¹ are each individually selected from the group of hydrogen, methyl, ethyl, n-propyl, iso-propyl, n-butyl, iso-butyl, sec-butyl, tert-butyl, and n-pentyl;
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
R²³ and R²⁴ are each individually selected from the group of methyl and ethyl.

Embodiment 11B. The method of Embodiment 11, wherein
R¹² and R¹³ are each individually selected from the group of hydrogen and methyl;
R¹⁴ is NH₂;
R¹⁵ is selected from the group of C(O)NR¹⁹R²⁰ and C(O)NHCH₃;
R¹⁶ is selected from the group of hydrogen and methyl;
R¹⁹, and R²⁰ are each individually selected from the group of hydrogen, methyl, ethyl, n-propyl, iso-propyl, n-butyl, iso-butyl, sec-butyl, tert-butyl, and n-pentyl;
Y is CN;
R¹ and R² are each individually selected from the group of hydrogen and methyl;
R³ is NH₂;
R⁴ is selected from the group of C(O)NR¹⁰R¹¹ and C(O)NHCH₃;
R⁵ is selected from the group of hydrogen and methyl;
R¹⁰, and R¹¹ are each individually selected from the group of hydrogen, methyl, ethyl, n-propyl, iso-propyl, n-butyl, iso-butyl, sec-butyl, tert-butyl, and n-pentyl;
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
R²³ and R²⁴ are each individually selected from the group of methyl and ethyl.

Embodiment 11C. The method of Embodiment 11, wherein
R¹² is hydrogen;
R¹³ is methyl;
R¹⁴ is NH₂;
R¹⁵ is selected from the group of C(O)NR¹⁹R²⁰ and C(O)NHCH₃;
R¹⁶ is hydrogen;
R¹⁹, and R²⁰ are each individually selected from the group of hydrogen, methyl, ethyl, n-propyl, and iso-propyl;
Y is CN;
R¹ is hydrogen;
R² is methyl;
R³ is NH₂;
R⁴ is selected from the group of C(O)NR¹⁰R¹¹ and C(O)NHCH₃;
R⁵ is hydrogen;
R¹⁰, and R¹¹ are each individually selected from the group of hydrogen, methyl, ethyl, n-propyl, and iso-propyl;
X is selected from the group of halogens, fluoride, chloride, bromide, and iodide; and
R²³ and R²⁴ are each individually methyl.

Embodiment 12. The method of Embodiment 11, wherein subjecting the mixture to irradiation from at least one light source comprises subjecting the mixture to a light radiation having a wavelength in a range of from about 180 nm to about 450 nm, preferably from about 180 nm to about 420 nm, more preferably from about 360 nm to about 420 nm, even more preferably from about 360 nm to about 400 nm.

Embodiment 12A. The method of Embodiment 11 or Embodiment 12, wherein subjecting the mixture to irradiation from at least one light source comprises subjecting the mixture to a light radiation having a wavelength in a range of from about 360 nm to about 420 nm.

Embodiment 12B. The method of Embodiment 11 or Embodiment 12, wherein subjecting the mixture to irradiation from at least one light source comprises subjecting the mixture to a light radiation having a wavelength in a range of from about 360 nm to about 400 nm.

Embodiment 13. The method of Embodiment 11, wherein the solubility of the compound of Formula II in the solvent is lower than the solubility of the compound of Formula I in the solvent.

Embodiment 14. The method of Embodiment 11, wherein the cyano-containing compound is soluble in the solvent.

Embodiment 15. The method of Embodiment 11, wherein subjecting the mixture to irradiation from at least one light source comprises subjecting the mixture to a light radiation having a power in a range of from about 1 W to about 1000 W.

Embodiment 16. The method of Embodiment 11, wherein subjecting the mixture to irradiation from at least one light source comprises subjecting the mixture to irradiation from at least one light source for a time in a range of from 1 second to about 12 hours.

Embodiment 17. The method of Embodiment 11, wherein the cyano-containing compound is selected from the group of salts of cyanide, NaCN, KCN, metal salts of cyanide, K₃[Fe(CN)₆], Na₃[Fe(CN)]₆, CuCN, Zn(CN)₂, acetonitrile, HCN, tetrabutylammonium cyanide, tetraethylammonium cyanide, trimethylsilyl cyanide, acetone cyanohydrin, and combinations thereof.

Embodiment 18. The method of Embodiment 11, wherein the solvent is selected from the group of aqueous solvents, organic solvents, water, acetonitrile, dimethylformamide (DMF), acetone, dimethylacetamide (DMAc), dimethyl sulfoxide (DMSO), N-methylpyrrolidone (NMP), alcohols, methanol, ethanol, n-propanol, isopropanol, n-butanol, 2-butanol, t-butanol, and combinations thereof, preferably wherein the solvent is a mixture of water and acetonitrile or a mixture of water and t-butanol.

Embodiment 19. The method of Embodiment 11, wherein the compound of Formula II is

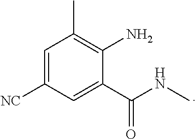

Embodiment 20. The method of Embodiment 11, wherein the compound of Formula I is

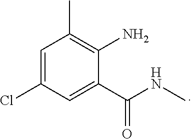

Embodiment 21. The method of any one of Embodiments 11 to 20 using the photochemical reaction system of any one of Embodiments 1 through 7 to prepare a compound of Formula II.

Embodiment 22. A method of preparing 2-amino-5-cyano-N,3-dimethyl-benzamide

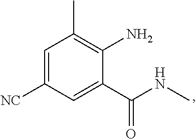

the method comprising:
I) forming a mixture comprising:
   a) 2-amino-5-chloro-N,3-dimethyl-benzamide

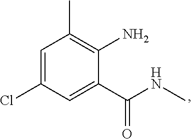

b) a cyano-containing compound; and
c) optionally a solvent; and
II) subjecting the mixture to irradiation from at least one light source.

Embodiment 23. The method of Embodiment 22, wherein the cyano-containing compound is selected from the group of salts of cyanide, NaCN, KCN, metal salts of cyanide, K₃[Fe(CN)₆], Na₃[Fe(CN)]₆, CuCN, Zn(CN)₂, acetonitrile, HCN, tetrabutylammonium cyanide, tetraethylammonium cyanide, trimethylsilyl cyanide, acetone cyanohydrin, and combinations thereof.

Embodiment 23A. The method of Embodiment 22, wherein the cyano-containing compound is selected from the group of NaCN, KCN, K₃[Fe(CN)₆], Na₃[Fe(CN)]₆, CuCN, and Zn(CN)₂.

Embodiment 23B. The method of Embodiment 22, wherein the cyano-containing compound is selected from the group of NaCN, KCN, CuCN, and Zn(CN)₂.

Embodiment 23C. The method of Embodiment 22, wherein the cyano-containing compound is NaCN.

Embodiment 24. The method of any one of Embodiments 22 through 23C, wherein the solvent is selected from the group of aqueous solvents, organic solvents, water, acetonitrile, dimethylformamide (DMF), acetone, dimethylacetamide (DMAc), dimethyl sulfoxide (DMSO), N-methylpyrrolidone (NMP), alcohols, methanol, ethanol, n-propanol, isopropanol, n-butanol, 2-butanol, t-butanol, and combinations thereof, preferably wherein the solvent is a mixture of water and acetonitrile or a mixture of water and t-butanol.

Embodiment 24A. The method of Embodiment 24, wherein the solvent is a mixture of water and acetonitrile or a mixture of water and t-butanol.

Embodiment 25. The method of any one of Embodiments 22 through 24A, wherein subjecting the mixture to irradiation from at least one light source comprises subjecting the mixture to a light radiation having a wavelength in a range of from about 360 nm to about 400 nm.

Embodiment 26. The method of any one of Embodiments 22 through 25 using the photochemical reaction system of any one of Embodiments 1 through 7.

Embodiment C1. A composition comprising:
(a) a compound of Formula II

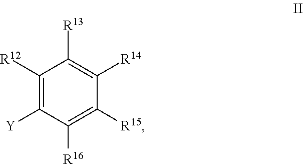

wherein:
R¹² and R¹³ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof;
R¹⁴ is selected from the group of amines, amides, carbamides, NH₂, NR¹⁷R¹⁸, and NR²¹COR²²;
R¹⁵ is selected from the group of amides, C(O)NR¹⁹R²⁰, and C(O)NHCH₃;
R¹⁶, R¹⁷, R¹⁸, R¹⁹, R²⁰, R²¹, and R²² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
Y is selected from the group of cyano-containing substituents and CN,
and
(b) a compound of Formula I

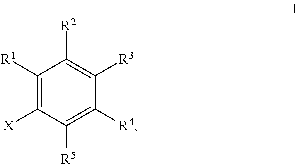

wherein:
   R¹ and R² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
   R³ is selected from the group of amines, amides, carbamides, NH₂, NR⁶R⁷, and NR⁸COR⁹;
   R⁴ is selected from the group of amides, C(O)NR¹⁰R¹¹, and C(O)NHCH₃;
   R⁵, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
   X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
   R²³ and R²⁴ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, C₁-C₆ haloalkyl, and C₁-C₆ fluoroalkyl.

Embodiment C2. The composition of Embodiment C1 optionally further comprising: (c) a compound of Formula III

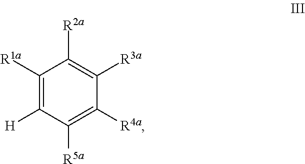

wherein:
   R^{1a} and R^{2a} are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
   R^{3a} is selected from the group of amines, amides, carbamides, NH₂, NR⁶R⁷, and NR⁸COR⁹;
   R^{4a} is selected from the group of amides, C(O)NR¹⁰R¹¹, and C(O)NHCH₃; and
   R^{5a}, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof.

Embodiment C2A. The composition of any one of Embodiments C1 to C2 further comprising: (c) the compound of Formula III.

Embodiment C3. The composition of any one of Embodiments C1 to C2A, wherein the compound of Formula II is

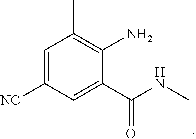

Embodiment C3A. The composition of any one of Embodiments C1 to C3, wherein the compound of Formula I is

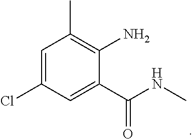

Embodiment C3B. The composition of any one of Embodiments C1 to C3A, wherein the compound of Formula III is

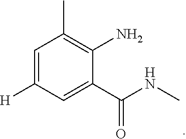

Embodiment C4. A composition comprising:
(a) 2-amino-5-cyano-N,3-dimethyl-benzamide

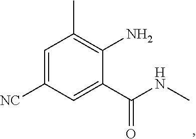

and
(b) 2-amino-5-chloro-N,3-dimethyl-benzamide

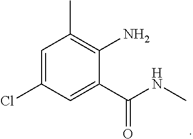

Embodiment C5. A composition comprising:
(a) 2-amino-5-cyano-N,3-dimethyl-benzamide

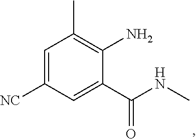

(b) 2-amino-5-chloro-N,3-dimethyl-benzamide

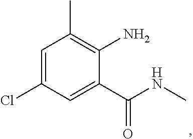

and
(c) 2-amino-N,3-dimethyl-benzamide

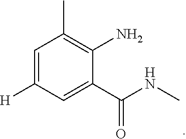

Embodiment C6. The composition of any one of Embodiments C1 to C5, wherein the molar ratio of the compound of Formula II to the compound of Formula I is from about 1:1 to about 99.9:0.1.

Embodiment C7. The composition of any one of Embodiments C1 to C6, wherein the molar ratio of the compound of Formula II to the compound of Formula I is from about 2:1 to about 99.9:0.1.

Embodiment C8. The composition of any one of Embodiments C1 to C7, wherein the molar ratio of the compound of Formula II to the compound of Formula I is from about 5:1 to about 99.9:0.1.

Embodiment C9. The composition of any one of Embodiments C1 to C8, wherein the molar ratio of the compound of Formula II to the compound of Formula I is from about 5:1 to about 99:1.

Embodiment C10. The composition of any one of Embodiments C1 to C9, wherein the molar ratio of the compound of Formula II to the compound of Formula I is from about 5:1 to about 50:1.

Embodiment C11. The composition of any one of Embodiments C1 to C10, wherein the molar ratio of the compound of Formula II to the compound of Formula I is from about 5:1 to about 30:1.

Embodiment C12. The composition of any one of Embodiments C2 to C11, wherein the molar ratio of the compound of Formula II to the compound of Formula III is from about 1:1 to about 99.9:0.1.

Embodiment C13. The composition of any one of Embodiments C2 to C12, wherein the molar ratio of the compound of Formula II to the compound of Formula III is from about 2:1 to about 99.9:0.1.

Embodiment C14. The composition of any one of Embodiments C2 to C13, wherein the molar ratio of the compound of Formula II to the compound of Formula III is from about 5:1 to about 99.9:0.1.

Embodiment C15. The composition of any one of Embodiments C2 to C14, wherein the molar ratio of the compound of Formula II to the compound of Formula III is from about 5:1 to about 99:1.

Embodiment C16. The composition of any one of Embodiments C2 to C15, wherein the molar ratio of the compound of Formula II to the compound of Formula III is from about 5:1 to about 50:1.

Embodiment C17. The composition of any one of Embodiments C2 to C16, wherein the molar ratio of the compound of Formula II to the compound of Formula III is from about 5:1 to about 30:1.

Embodiment C18. The composition of any one of Embodiments C1 to C17, wherein the composition is prepared by the method of any one of Embodiments 11 to 20.

In one aspect, a compound of Formula II is prepared according to a method represented by Scheme 1. The R groups are as defined anywhere in this disclosure.

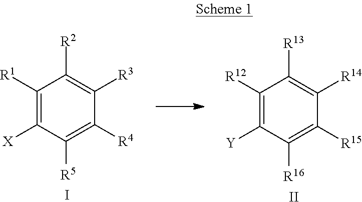

This aspect includes receiving in a receiving vessel of a photochemical reaction system a mixture including a compound of Formula I, a cyano-containing compound, and optionally a solvent; and then reacting the mixture in the photochemical reaction system to produce the compound of Formula II.

In many embodiments, reacting the mixture in the photochemical reaction system comprises subjecting the mixture to irradiation from at least one light source. Generally, the radiation may be of any suitable wavelength known in the art. In some embodiments, the radiation comprises ultraviolet radiation, visible radiation, or a combination thereof.

In some embodiments, the radiation has a minimum wavelength selected from 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, and 800 nm.

In some embodiments, the radiation has a maximum wavelength selected from 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, and 800 nm.

In some embodiments, the radiation has a wavelength in a range of from about 180 nm to about 450 nm. In some embodiments, the radiation has a wavelength in a range of from about 180 nm to about 400 nm. In some embodiments, the radiation has a wavelength in a range of from about 360 nm to about 420 nm. In some embodiments, the radiation has a wavelength in a range of from about 380 nm to about 400 nm.

Generally, the radiation may be of any suitable power known in the art. In many embodiments, the power depends on various factors and is non-essential. In some embodiments, the radiation has a power in a range of from about 1 W to about 1000 W.

Generally, the radiation may be applied for any suitable time known in the art. In many embodiments, the time depends on various factors and is non-essential. In some embodiments, the radiation is applied for a time in a range of from 1 second to about 12 hours.

Generally, the cyano-containing compound may be any suitable cyano-containing compound known in the art. In some embodiments, the cyano-containing compound is selected from the group of salts of cyanide, NaCN, KCN, metal salts of cyanide, K₃[Fe(CN)₆], Na₃[Fe(CN)]₆, CuCN, Zn(CN)₂, acetonitrile, HCN, tetrabutylammonium cyanide, tetraethylammonium cyanide, trimethylsilyl cyanide, acetone cyanohydrin, and combinations thereof.

Generally, the solvent may be any suitable solvent known in the art. In some embodiments, the solvent is selected from the group of aqueous solvents, organic solvents, water, acetonitrile, dimethylformamide (DMF), acetone, dimethylacetamide (DMAc), dimethyl sulfoxide (DMSO), N-methylpyrrolidone (NMP), alcohols, methanol, ethanol, n-propanol, isopropanol, n-butanol, 2-butanol, t-butanol, and combinations thereof. In some preferred embodiments, the solvent is a mixture of water and acetonitrile or a mixture of water and t-butanol.

In some embodiments, when the reaction occurs in a loop reactor system, the solubility of the compound of Formula II in the solvent is lower than the solubility of the compound of Formula I in the solvent. In these embodiments, the differences in solubility enable cycling of the reaction by allowing the compound of Formula II to precipitate out of solution and be collected. Such collection may occur with the use of a filter.

In some embodiments, when the reaction occurs in a loop reactor system, the cyano-containing compound is soluble in the solvent. In these embodiments, the solubility of the cyano-containing compound enables cycling of the reaction by avoiding precipitation of the cyano-containing compound.

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application. Ambient or room temperature is defined as about 20-25° C. The term HPLC means high performance liquid chromatography.

### Example 1

### Preparation of 2-amino-5-cyano-N,3-dimethyl-benzamide

To a 25 mL reactor was added 0.3 g 2-amino-5-chloro-N,3-dimethyl-benzamide, 0.3 g sodium cyanide (NaCN), 7.5 g acetonitrile, and 7.5 g water at ambient temperature. The mixture was stirred under 370 nm light irradiation for 5 h. HPLC analysis showed 94.9% 2-amino-5-cyano-N,3-dimethyl-benzamide and 2.4% 2-amino-5-cyano-N,3-dimethyl-benzamide. Weight analysis showed a reaction yield of 80%.

### Example 2

### Preparation of 2-amino-5-cyano-N,3-dimethyl-benzamide

To a 25 mL reactor was added 0.8 g 2-amino-5-chloro-N,3-dimethyl-benzamide, 0.8 g sodium cyanide (NaCN), 7.5 g acetonitrile, and 7.5 g water at 60° C. The mixture was stirred under 370 nm light irradiation for 5 h. HPLC analysis showed 95.5% 2-amino-5-cyano-N,3-dimethyl-benzamide and 1.2% 2-amino-5-cyano-N,3-dimethyl-benzamide. Weight analysis showed a reaction yield of 76%.

### Example 3

### Preparation of 2-amino-5-cyano-N,3-dimethyl-benzamide Using Different Wavelengths of Irradiation

To a 25 mL reactor is added 0.3 g 2-amino-5-chloro-N,3-dimethyl-benzamide, 0.3 g sodium cyanide (NaCN), 7.5 g acetonitrile, and 7.5 g water at ambient temperature. The mixture was stirred under different wavelengths of light irradiation for 4 h. HPLC analysis showed different conversions depending on the wavelength of irradiation, as indicated in Table 1 below.

TABLE 1

| Wave- | Irradiation | 2-amino-5-cyano-N,3- | 2-amino-5-chloro-N,3- |
|---|---|---|---|
| length | time | dimethyl-benzamide | dimethyl-benzamide |
| (nm) | (h) | (%) | (%) |
| 255 | 4 | 3.73 | 96.27 |
| 285 | 4 | 8.01 | 91.99 |
| 305 | 4 | 12.05 | 87.95 |
| 370 | 4 | 90.94 | 4.30 |
| 390 | 4 | 50.30 | 47.66 |

### Example 4

### Preparation of 2-amino-5-cyano-N,3-dimethyl-benzamide Using Different Wavelengths of Irradiation

To a 25 mL reactor is added 0.3 g 2-amino-5-chloro-N,3-dimethyl-benzamide, 0.3 g sodium cyanide (NaCN), 7.5 g acetonitrile, and 7.5 g water at ambient temperature. The mixture was stirred under different wavelengths of light irradiation for 3 h. HPLC analysis showed different conversions depending on the wavelength of irradiation, as indicated in Table 2 below.

TABLE 2

| | | 2-amino-5-cyano-N,3- | 2-amino-5-chloro-N,3- | 2-amino-N,3- |
|---|---|---|---|---|
| | | dimethyl-benzamide | dimethyl-benzamide | dimethyl-benzamide |
| Wavelength | Irradiation time | (product) | (starting material) | (by-product) |
| (nm) | (h) | (%) | (%) | (%) |
| 185 | 3 | 5.5 | 94.5 | Not detected |
| 255 | 3 | 4.4 | 95.6 | Not detected |
| 285 | 3 | 4.4 | 95.4 | 0.2 |
| 305 | 3 | 7.7 | 91.7 | 0.6 |
| 370 | 3 | 88.5 | 4.4 | 3.3 |
| 390 | 3 | 83.0 | 8.9 | 4.6 |
| 427 | 3 | 11.9 | 86.2 | 0.8 |
| 440 | 3 | 2.2 | 97.9 | Not detected |

### Example 5

### Preparation of 2-amino-5-cyano-N,3-dimethyl-benzamide Using Different Wavelengths of Irradiation

To each quartz vessel of a parallel UV reactor is added 0.3 g 2-amino-5-chloro-N,3-dimethyl-benzamide, 0.3 g sodium cyanide (NaCN), 7.5 g acetonitrile, and 7.5 g water at ambient temperature. The mixtures were stirred under different wavelengths of light irradiation for 3 h. HPLC analysis showed different conversions depending on the wavelength of irradiation, as indicated in Table 2 below.

TABLE 2

| | | 2-amino-5-cyano-N,3- | 2-amino-5-chloro-N,3- | 2-amino-N,3- |
|---|---|---|---|---|
| | | dimethyl-benzamide | dimethyl-benzamide | dimethyl-benzamide |
| Wavelength | Irradiation time | (product) | (starting material) | (by-product) |
| (nm) | (h) | (%) | (%) | (%) |
| 250-260 | 3 | 12.1 | 85.9 | 0.6 |
| 260-270 | 3 | 20.8 | 78.2 | 1.2 |
| 270-280 | 3 | 21.7 | 77.4 | 1.1 |
| 280-290 | 3 | 24.2 | 74.8 | 1.3 |
| 290-300 | 3 | 40.2 | 57.9 | 2.3 |
| 300-310 | 3 | 56.0 | 40.8 | 2.7 |
| 310-320 | 3 | 23.6 | 73.1 | 1.2 |
| 320-330 | 3 | 19.9 | 76.9 | 1.0 |
| 330-340 | 3 | 21.6 | 75 | 1.1 |
| 340-350 | 3 | 24.1 | 72.2 | 1.2 |
| 365-370 | 3 | 86.00 | 3.4 | 6.4 |

### Example 6

### Preparation of 2-amino-5-cyano-N,3-dimethyl-benzamide Using a Loop Reactor System

To a first 500 mL reactor with a filter plate was added 50 g 2-amino-5-chloro-N,3-dimethyl-benzamide, 36 g sodium cyanide (NaCN), 228 g t-butanol, and 686 g water at room temperature. The solution under the filter plate was piped to a photo reactor by a first pump. A reaction was conducted under 370 nm light irradiation. The resultant warm reaction liquid was cooled to room temperature and transferred to a second 500 mL reactor with a filter plate. 2-Amino-5-cyano-N,3-dimethyl-benzamide was precipitated in the second reactor. The mother liquid (i.e. filtrate) was transferred to the first reactor by a second pump. The system was run until all the 2-amino-5-cyano-N,3-dimethyl-benzamide disappeared. HPLC analysis showed conversion of 2-amino-5-cyano-N,3-dimethyl-benzamide of greater than 98%. Weight analysis showed a reaction yield of 82%.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

When a group contains a substituent which can be hydrogen, for example R⁴, then, when this substituent is taken as hydrogen, it is recognized that this is equivalent to said group being unsubstituted.

The term “cyano-containing compound” includes, without limitation, a compound including a functional group comprising a nitrile bond (—C≡N).

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

What is claimed is:

1. A method of preparing a compound of Formula II 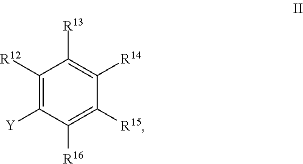
wherein:
R¹² and R¹³ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof;
R¹⁴ is selected from the group of amines, amides, carbamides, NH₂, NR¹⁷R¹⁸, and NR²¹COR²²;
R¹⁵ is selected from the group of amides, C(O)NR¹⁹R²⁰, and C(O)NHCH₃;
R¹⁶, R¹⁷, R¹⁸, R¹⁹, R²⁰, R²¹, and R²² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
Y is selected from the group of cyano-containing substituents and CN,
the method comprising:
I) forming a mixture comprising:
a) a compound of Formula I 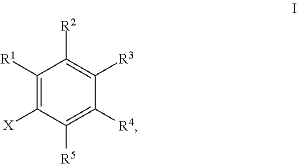
wherein:
R¹ and R² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
R³ is selected from the group of amines, amides, carbamides, NH₂, NR⁶R⁷, and NR⁸COR⁹;
R⁴ is selected from the group of amides, C(O)NR¹⁰R¹¹, and C(O)NHCH₃;
R⁵, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl and isomers; and
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide;
R²³ and R²⁴ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, C₁-C₆ haloalkyl, and C₁-C₆ fluoroalkyl;
b) a cyano-containing compound; and
c) optionally a solvent; and
II) subjecting the mixture to irradiation from at least one light source.

2. The method of claim 1, wherein subjecting the mixture to irradiation from at least one light source comprises subjecting the mixture to a light radiation having a wavelength in a range of from about 180 nm to about 450 nm, preferably from about 180 nm to about 420 nm, more preferably from about 360 nm to about 420 nm, even more preferably from about 360 nm to about 400 nm.

3. The method of claim 1, wherein the solubility of the compound of Formula II in the solvent is lower than the solubility of the compound of Formula I in the solvent.

4. The method of claim 1, wherein the cyano-containing compound is soluble in the solvent.

5. The method of claim 1, wherein subjecting the mixture to irradiation from at least one light source comprises subjecting the mixture to a light radiation having a power in a range of from about 1 W to about 1000 W.

6. The method of claim 1, wherein subjecting the mixture to irradiation from at least one light source comprises subjecting the mixture to irradiation from at least one light source for a time in a range of from 1 second to about 12 hours.

7. The method of claim 1, wherein the cyano-containing compound is selected from the group of salts of cyanide, NaCN, KCN, metal salts of cyanide, K₃[Fe(CN)₆], Na₃[Fe(CN)]₆, CuCN, Zn(CN)₂, acetonitrile, HCN, tetrabutylammonium cyanide, tetraethylammonium cyanide, trimethylsilyl cyanide, acetone cyanohydrin, and combinations thereof.

8. The method of claim 1, wherein the solvent is selected from the group of aqueous solvents, organic solvents, water, acetonitrile, dimethylformamide (DMF), acetone, dimethylacetamide (DMAc), dimethyl sulfoxide (DMSO), N-methylpyrrolidone (NMP), alcohols, methanol, ethanol, n-propanol, isopropanol, n-butanol, 2-butanol, t-butanol, and combinations thereof, preferably wherein the solvent is a mixture of water and acetonitrile or a mixture of water and t-butanol.

9. The method of preparing a compound of Formula II of claim 1, wherein the compound of Formula II is 2-amino-5-cyano-N,3-dimethyl-benzamide 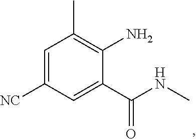
the method comprising:
I) forming a mixture comprising:
a) a compound of Formula I, wherein the compound of Formula I is 2-amino-5-chloro-N,3-dimethyl-benzamide 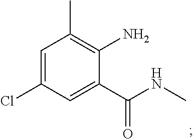
b) a cyano-containing compound; and
c) optionally a solvent; and
II) subjecting the mixture to irradiation from at least one light source wherein the light radiation has a wavelength in a range of from about 360 nm to about 400 nm.

10. The method of claim 1, wherein the subjecting the mixture to irradiation from at least one light source is performed using a photochemical reaction system comprising:
a loop reactor system comprising:
a receiving vessel configured to receive a mixture;
a photochemical reaction vessel configured to subject the mixture to irradiation from at least one light source; and
a precipitation vessel configured to receive the irradiated mixture; and
optionally a further reaction system component selected from the group of fluid transfer systems, stripping systems, filtration systems, drying systems, stirring systems, temperature control systems, and combinations thereof.

11. The method of claim 10, wherein the loop reactor system is configured to cycle the mixture at least once through the receiving vessel, the photochemical reaction vessel, and the precipitation vessel.

12. The method of claim 10, wherein the photochemical reaction system comprises at least one fluid transfer system configured to:
transfer the mixture from the receiving vessel to the photochemical reaction vessel;
transfer the mixture from the photochemical reaction vessel to the precipitation vessel; and/or
transfer the mixture from the precipitation vessel to the receiving vessel.

13. The method of claim 10, wherein the photochemical reaction system comprises a stripping system, a stirring system, a filtration system, a drying system, a temperature control system, or a combination thereof.

14. The method of claim 10, wherein the photochemical reaction vessel is configured to subject the mixture to irradiation from a plurality of light sources.

15. The method of claim 10, wherein the photochemical reaction vessel is configured to subject the mixture to a light radiation having a wavelength in a range of from about 180 nm to about 450 nm, preferably from about 180 nm to about 420 nm, more preferably from about 360 nm to about 420 nm, even more preferably from about 360 nm to about 400 nm.

16. The method of claim 10, wherein at least one of the receiving vessel and the precipitation vessel comprises a filter.

17. A composition comprising:
(a) a compound of Formula II 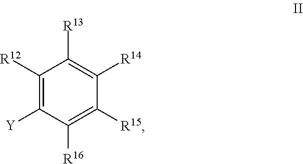
wherein:
R¹² and R¹³ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof;
R¹⁴ is selected from the group of amines, amides, carbamides, NH₂, NR¹⁷R¹⁸, and NR²¹COR²²;
R¹⁵ is selected from the group of amides, C(O)NR¹⁹R²⁰, and C(O)NHCH₃;
R¹⁶, R¹⁷, R¹⁸, R¹⁹, R²⁰, R²¹, and R²² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
Y is selected from the group of cyano-containing substituents and CN,
and
(b) a compound of Formula I 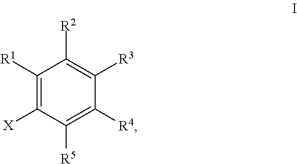
wherein:
R¹ and R² are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
R³ is selected from the group of amines, amides, carbamides, NH₂, NR⁶R⁷, and NR⁸COR⁹;
R⁴ is selected from the group of amides, C(O)NR¹⁰R¹¹, and C(O)NHCH₃;
R⁵, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof; and
X is selected from the group of leaving groups, trifluoromethanesulfonate (TfO), tosylate (TsO), p-tosylate (p-TsO), mesylate (MsO), diethyl phosphate (PO₄Et₂), sulfonates (—OSO₂R²³), carboxylates (—OCOR²⁴), diazonium, halogens, fluoride, chloride, bromide, and iodide; and
R²³ and R²⁴ are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, C₁-C₆ haloalkyl, and C₁-C₆ fluoroalkyl.

18. The composition of claim 17 further comprising:
(c) a compound of Formula III 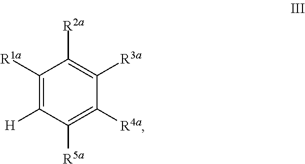
wherein:
R^{1a} and R^{2a} are each individually selected from the group of hydrogen, substituted or unsubstituted linear alkyl, substituted or unsubstituted C₁-C₆ linear alkyl, substituted or unsubstituted branched alkyl, substituted or unsubstituted C₃-C₆ branched alkyl, methyl, ethyl, n-propyl, iso-propyl, n-butyl, iso-butyl, sec-butyl, tert-butyl, n-pentyl and isomers;
R^{3a} is selected from the group of amines, amides, carbamides, NH₂, NR⁶R⁷, and NR⁸COR⁹;
R^{4a} is selected from the group of amides, C(O)NR¹⁰R¹¹, and C(O)NHCH₃; and
R^{5a}, R⁶, R⁷, R⁸, R⁹, R¹⁰, and R¹¹ are each individually selected from the group of hydrogen, substituted or unsubstituted linear alkyl, substituted or unsubstituted C₁-C₆ linear alkyl, substituted or unsubstituted branched alkyl, and substituted or unsubstituted C₃-C₆ branched alkyl, methyl, ethyl, n-propyl, iso-propyl, n-butyl, iso-butyl, sec-butyl, tert-butyl, n-pentyl, and isomers thereof.

19. The composition of claim 17 wherein
the compound of Formula II is 2-amino-5-cyano-N,3-dimethyl-benzamide, and
the compound of Formula I is 2-amino-5-chloro-N,3-dimethyl-benzamide.

20. The composition of claim 18 wherein
the compound of Formula II is 2-amino-5-cyano-N,3-dimethyl-benzamide,
the compound of Formula I is 2-amino-5-chloro-N,3-dimethyl-benzamide, and
the compound of Formula III is 2-amino-N,3-dimethyl-benzamide.
